(19) United States
(12) Patent Application Publication
(10) Pub. No.: US 2007/0087434 A1
(43) Pub. Date: Apr. 19, 2007

(54) ANTIVIRAL ARTIFICIAL CELL

(75) Inventors: Yujiro Naruse, Yokohama-shi (JP); Kazuhiko Itaya, Yokohama-shi (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: KABUSHIKI KAISHA TOSHIBA, Tokyo (JP)

(21) Appl. No.: 11/535,627

(22) Filed: Sep. 27, 2006

(30) Foreign Application Priority Data
Sep. 29, 2005 (JP) P2005-285409

(51) Int. Cl. C12N 7/00; C12N 5/00; C12N 7/01; C12N 5/02

(52) U.S. Cl. 435/325; 435/235.1

(57) ABSTRACT

An antiviral artificial cell includes: an artificial cytoskeleton, an artificial cytomembrane wrapping the artificial cytoskeleton, and a nanoparticle rotatably retained on the artificial cytomembrane and having a surface for capturing a virus.

### CROSS-REFERENCE TO RELATED APPLICATIONS

The entire disclosure of Japanese Patent Application No. 2005-285409 filed on Sep. 29, 2005 including specification, claims, drawings and abstract is incorporated herein by reference in its entirety.

### BACKGROUND OF THE INVENTION

1. Field of the Invention

This invention relates to an antiviral artificial cell to be used as an antiviral drug for therapy of disorder and disease attributable to a virus and to be used for a biofilter for removing the virus.

2. Description of the Related Art

An antiviral drug used for therapy of a viral disorder or a viral infection is generally a chemical substance such as ribavirin and exhibits an antiproliferative effect by acting on mRNA or the like having an important role in a virus proliferation process.

However, such chemical substance has problems such as an adverse effect, possibility of being the cause of a drug resistant virus, and long term product development for the newly emerged virus.

Therefore, as a therapeutic method without the use of the antiviral drug, a method wherein blood containing a virus is extracted from a human body to outside of the human body, and the virus is separated from normal blood components to return the normal blood components to the human body as well as to disinfect the separated virus has been studied (see, for example, JP-A-6-183998 (KOKAI)). Also, an inorganic core liposome which is obtainable by coating inorganic fine particles with liposome and providing a functional group acting on a virus on a surface of the liposome has been developed (see, for example, WO93/26019).

However, with the method of exteriorizing blood, it is difficult to perform the treatment directly on affected cells. Further, since the conventional core liposome is nothing more than that having on its surface the functional group for capturing the virus, the inorganic liposome can fail to satisfactorily perform neutralization of the virus and has difficulty in externally controlling the neutralization of virus.

### SUMMARY OF THE INVENTION

This invention has been accomplished in view of the above-described circumstances, and provides an antiviral artificial cell which is capable of reliably performing neutralization of a virus and which enables an externally control of the neutralization of virus.

### BRIEF DESCRIPTION OF THE DRAWINGS

Exemplary embodiments may be described in detail with reference to the accompanying drawings, in which:

FIG. 1 is a diagram showing a schematic structure of an antiviral artificial cell according to one embodiment of the invention;

FIGS. 2A and 2B are diagrams showing a rotation mode of the nanoparticle and a mode of attachment/detachment of a virus;

FIGS. 3A-3C are diagrams showing a method of wrapping an artificial cytoskeleton with an artificial cytomembrane (artificial cell membrane);

FIGS. 4A-4C are diagrams showing one example of preparation method of a nanoparticle having a concavo-convex part.

FIG. 5 is a diagram showing another example of preparation method of a nanoparticle having a concavo-convex part.

### DETAILED DESCRIPTION OF THE INVENTION

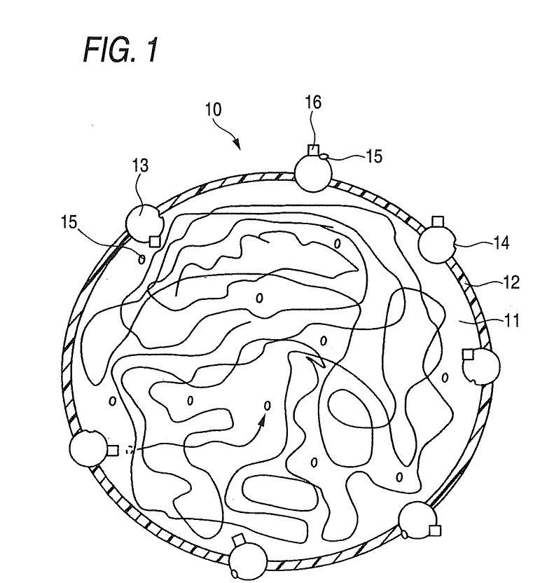

Hereinafter, embodiments of this invention will be described in detail with reference to accompanying drawings. Shown in FIG. 1 is a schematic structure of an antiviral artificial cell 10. This antiviral artificial cell 10 has an artificial cytoskeleton 11, an artificial cytomembrane (artificial cell membrane) 12 for wrapping the artificial cytoskeleton 11, and a nanoparticle 13 rotatably retained by the cytomembrane 12.

As the artificial cytoskeleton 11, an electromagnetic wave absorber may suitably be used. With the use of the electromagnetic wave absorber, the artificial cytoskeleton 11 generates heat upon irradiation of the antiviral artificial cell 10 with an electromagnetic wave to neutralize a virus 15 entrapped by the artificial cytoskeleton 11. The entrapment of the virus 15 by the artificial cytoskeleton 11 will be described later in this specification.

As the electromagnetic wave absorber, a carbon nanotube may preferably be used, and it is appropriate to use a sponge-like carbon nanotube containing moisture as the electromagnetic wave absorber. Also, the artificial cytoskeleton 11 may be obtained by mixing a metal nanowire with an ordinary fiber. In the case where the artificial cytoskeleton 11 is formed of a magnetizable material, it is possible to attract the antiviral artificial cell 10 into an affected area inside a human body or the like with the use of an external DC magnetic field by magnetizing the artificial cytoskeleton 11.

The cytomembrane 12 may be a phospholipid polymer, for example.

A material to be used for the nanoparticle 13 is not limited, and, for example, a metal, a semiconductor, a resin, or a ceramic may be used as the material. Since it is difficult for an ordinary nanoparticle as it is to capture a virus on its surface, it is preferable that a surface of the nanoparticle 13 is physically modified for the purpose of facilitating the virus capturing, specifically, the surface may preferably be provided with a concavo-convex part 14 for the virus 15 to be fitted into. With the concavo-convex part 14, it is possible to increase possibility of capturing the virus 15. It is not always necessary to form the concavo-convex part 14 uniformly on the surface of the nanoparticle 13.

The nanoparticle 13 rotates by external physical excitation. As the physical excitation, oscillation (oscillatory wave) may suitably be employed. When the nanoparticle 13 is rotated after the virus 15 is captured at the concavo-convex part 14 provided on the surface of the nanoparticle 13, the virus 15 moves to the inside of the artificial cytomembrane 12, so that the virus 15 is detached from the concavo-convex part 14 due to interaction with the artificial cytoskeleton 11 (e.g. friction or capillary phenomenon) to be entrapped by the artificial cytoskeleton 11. After that, the artificial cytoskeleton 11 is heated to neutralize the virus 15.

In the case of rotating the nanoparticle 13 by oscillation, the nanoparticle 13 may preferably be decentered (i.e. the gravity center is shifted from the center) since it is easier to cause the rotation when the nanoparticle 13 is decentered. As a method of decentering the nanoparticle 13, nanoparticles (hereinafter referred to as decentering nanoparticles) 16 having the size smaller than that of the nanoparticle 13 are fixed non-uniformly on the surface of the nanoparticle 13. Like the concavo-convex part 14 formed on the nanoparticle 13, each of the decentering nanoparticles 16 may have a concavo-convex part for facilitating the virus capturing. Also, as another example of the method of decentering the nanoparticle 13, the concavo-convex parts 14 may be formed non-uniformly on the surface of the nanoparticle 13 or an ion having a mass different from that of the nanoparticle 13 may be non-uniformly injected into the nanoparticle 13.

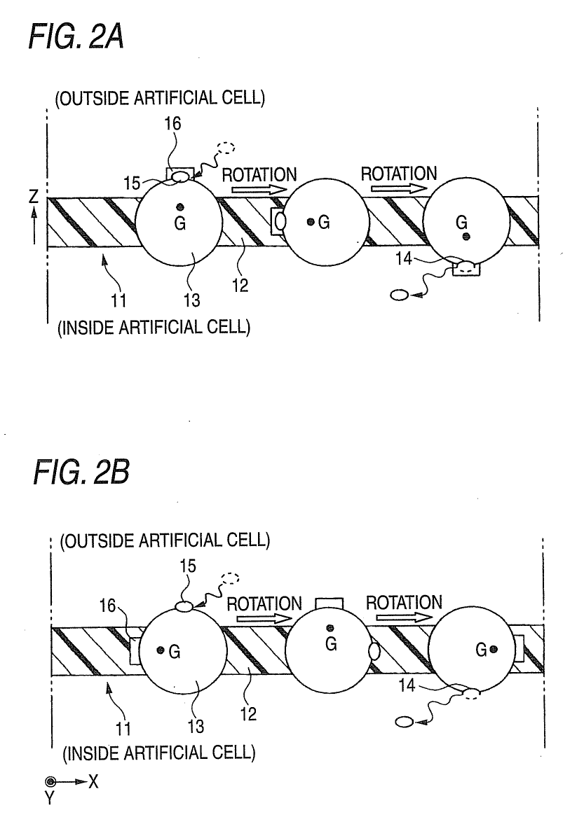

Shown in FIGS. 2A and 2B are schematic illustrations of a rotation mode of the nanoparticle 13 and a mode of attachment/detachment of the virus 15. When the antiviral artificial cell 10 is oscillated by externally applying an oscillatory wave or the like thereto, ruffling oscillation of the artificial cytomembrane 12 is caused so that the gravity center of the nanoparticle 13 present inside the artificial cytomembrane 12 receives acceleration in a predetermined direction represented by a three dimensional vector. The oscillation components can be divided into a z-component of FIG. 2A perpendicular to the artificial cytomembrane 12 and an xy-component of FIG. 2B parallel to the artificial cytomembrane 12 (y component is perpendicular to the drawing sheet). In the perpendicular mode (z-component oscillation) of FIG. 2A, the virus 15 is captured on the surface adjacent to the gravity center G of the nanoparticle 13, and, the nanoparticle 13 rotates by 180 degrees to release the virus 15 in the artificial cytoskeleton 11 due to interaction with the artificial cytoskeleton 11. In the parallel mode (xy-component oscillation) of FIG. 2B, the virus 15 is captured on the surface shifted by 90 degrees from a radial direction connecting the gravity center G to the center of the nanoparticle 13, and, the nanoparticle 13 rotates by 180 degrees to release the virus 15 in the artificial cytoskeleton 11 due to interaction with the artificial cytoskeleton 11.

Hereinafter, conditions under which the nanoparticle 13 can rotate in a state where it is held by the artificial cytomembrane 12 will be described. In the case where a centrifugal force of the gravity center G caused by the rotation of the nanoparticle 13 is Fc, a vector force generated by the ruffling motion of the artificial cytomembrane 12 is Fv, and a force of the artificial cytomembrane 12 for binding the nanoparticle 13 is Fb, the conditions under which the decentered nanoparticle 13 is not detached from the artificial cytomembrane 12 are given by the following expression (1). It is necessary to decide an angular frequency oof the oscillation to be applied to the antiviral artificial cell 10.
Fc+Fv<Fb (1)

Also, it is necessary that the rotation of the nanoparticle 13 be maintained in synchronization with the external oscillation. Therefore, in the case where motion energy given to the nanoparticle 13 per external oscillation cycle is En, an energy loss due to rotation friction of the nanoparticle 13 is Er(ω), an energy loss due to parallel oscillation in the artificial cytomembrane 12 is Ep(ω), the following expression (2) must be satisfied.
Er(ω)+Ep(ω)<En (2)

Since the energy losses Er(ω) and Ep(ω) are generally increased with an increase in angular frequency ω, it is necessary to decide the upper limit of the angular frequency ω so as to satisfy the expression (2).

The antiviral artificial cell 10 having the above-described constitution is injected into a treatment site by oral administration or intravenous injection (instillation) or applied on an affected area. Thus, a virus is captured by the nanoparticle 13. After that, oscillation of an ultrasonic wave or the like is applied on the antiviral artificial cell 10 to rotate the nanoparticle 13 for the entrapment of the virus by the artificial cytoskeleton 11. Then, an electromagnetic wave is applied on the antiviral artificial cell 10 to heat the artificial cytoskeleton 11. Thus, protein and RNA/DNA of the virus are modified so that the virus is neutralized.

The antiviral artificial cell 10 is used not only for the treatment of affected area of a human body or an animal and the virus removal from blood or biologic fluid but also for a filter of an air conditioner or a water purifier which is required to capture and neutralize viruses. For instance, with a system in which the antiviral artificial cell 10 is supported by a nonwoven cloth or an active carbon forming the filter and oscillation and an electromagnetic wave are applied at predetermined interval, it is possible not only to remove viruses from the air and water but also to keep the filter clean.

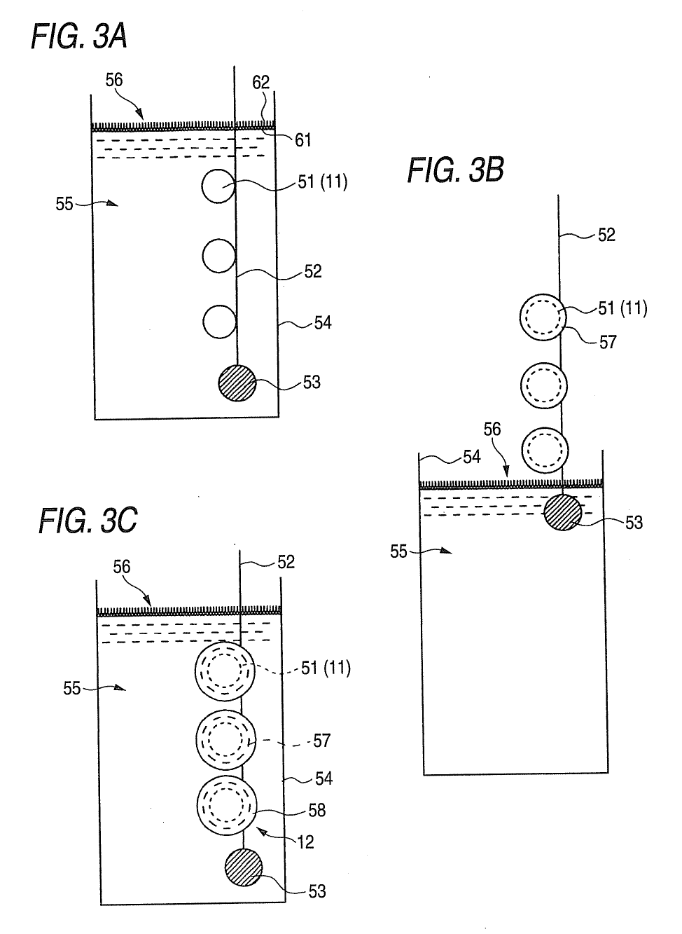

Hereinafter, a method for producing the antiviral artificial cell 10 will be described. Shown in FIGS. 3A-3C are schematic illustrations of a method of wrapping the artificial cytoskeleton 11 with the artificial cytomembrane 12. For instance, a carbon nanotube containing moisture is formed into spheres each having a diameter of 10 to 30 μm, and the spheres 51 are aligned on and fixed to a fine thread 52 having a diameter smaller than that of the sphere 51 with a biocompatible adhesive. The spheres 51 ultimately become the artificial cytoskeleton 11. A spindle 53 is attached to a lower end of the thread 52, and the spheres 51 are dipped into pure water 55 contained in a vessel 54 (FIG. 3A). A dispersion or the like for retaining the spheres 51 in water stably may be added to the pure water 55.

After that, a phospholipid polymer film 56 is formed on a surface of the pure water 55. Since the phospholipid polymer has a molecular structure including a hydrophilic group 61 and a hydrophobic group 62, the hydrophilic group 61 sinks down below a surface of the pure water 55, while the hydrophobic group 62 is projected out of the surface of the pure water 55. Then, the thread 52 is pulled up so that the spheres 51 which have been directly under the phospholipid polymer film 56 are drawn out of the pure water 55. Thus, the hydrophilic group 61 of the phospholipid polymer is bonded to the surfaces of the spheres 51, so that a first phospholipid polymer film 57 having the hydrophobic group projecting radially is formed (FIG. 3B). After that, the spheres 51 are dipped into the pure water 55 again, so that a second phospholipid polymer film 58 in which the hydrophobic group 62 is positioned inside and the hydrophilic group 61 is positioned outside is formed to cover the first phospholipid polymer film 57 (FIG. 3C). The thus formed phospholipid polymers film having the two-layer structure is the artificial cytomembrane 12. After forming the artificial cytomembrane 12 on the artificial cytoskeleton 11, the phospholipid polymer film 56 on the surface of the pure water 55 is removed.

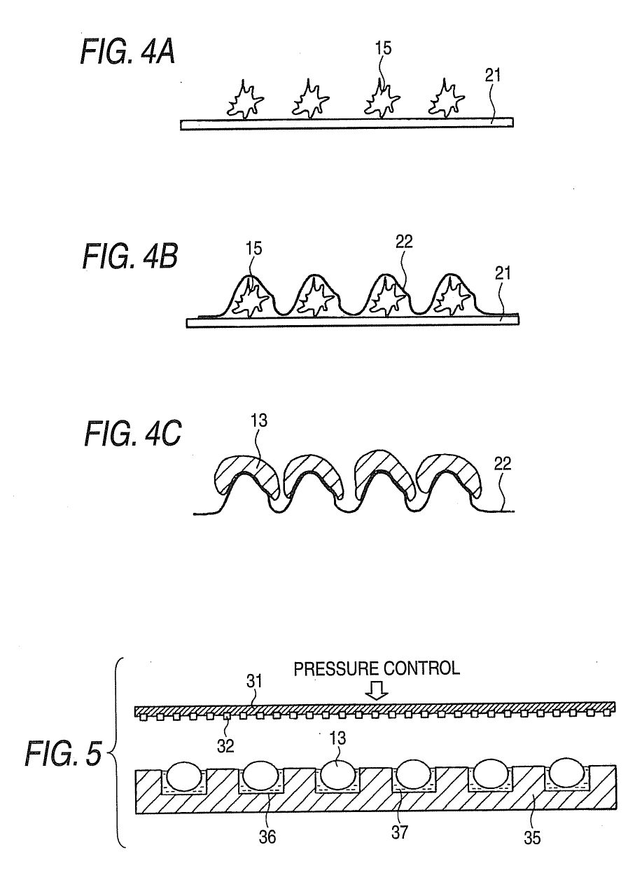

The nanoparticle 13 having the concavo-convex part 14 is prepared separately from the artificial cytoskeleton 11 and the artificial cytomembrane 12. Shown in FIGS. 4A-4C are schematic illustrations of one example of preparation method of the nanoparticle 13. As shown in FIG. 4A, the virus 15 is fixed to a surface of a glass substrate 21 by quick freezing. Then, as shown in FIG. 4B, a platinum replica 22 is formed by subjecting the glass substrate 21 to platinum vapor deposition. After that, as shown in FIG. 4C, gold or ceramic are deposited on the platinum replica 22 by sputtering or the like to obtain the nanoparticle 13 having a dent at which the virus 15 is easily captured.

Shown in FIG. 5 is a schematic illustration of another example of preparation method of the nanoparticle having the concavo-convex part 14. A nano-template 31 having projections 32 each having the shape of the virus 15 is prepared. The nano-template 31 can be formed by the use of the platinum replica 22 shown in FIG. 2. A substrate having grooves 36 is prepared, and the nanoparticles 13 (with or without the concavo-convex part 14) are placed in the grooves 36. A glass substrate or a semiconductor substrate may be used as the substrate 35, and the grooves 36 may be formed by employing a semiconductor production process such as photolithography and etching. It is preferable that a depth of each of the grooves 36 is smaller than a shorter diameter of the nanoparticle 13, and that a width thereof is longer than a longer diameter of the nanoparticle 13. In order to suppress fixation of the nanoparticles 13 to the substrate 35, the grooves 36 may preferably be filled with a liquid 37 functioning as a mold release agent, such as pure water and an organic solvent. Then, a temperature of the substrate 35 is retained at a predetermined value, and the nano-template 31 is pressed against the substrate 35 with a predetermined pressure, so that the projections 32 are transcribed onto the nanoparticles 13, thereby obtaining the nanoparticles 13 each having the concavo-convex part 14. Such nano-press technology is suitably used as a method of forming the concavo-convex part on the nanoparticle made from a thermoplastic resin.

The decentering nanoparticles 16 separately prepared are fixed to the thus prepared nanoparticle 13 by, for example, a ultrasonic thermal adhesion method (the nanoparticles 13 and 16 are mixed in an aqueous solution followed by ultrasonic wave application, so that the resin of the nanoparticle 13 is melted by collision of the nanoparticles 13 and 16 to adhere the nanoparticles 16 to the nanoparticle 13). The adhesion of the decentering nanoparticles 16 to the nanoparticle 13 may be performed before forming the concavo-convex part 14 on the nanoparticle 13.

The thus prepared nanoparticle 13 is thrown into the pure water 55, followed by ultrasonic wave application with stirring. Thus, the nanoparticle 13 is held by the artificial cytomembrane 12 to obtain the antiviral artificial cell 10.

According to the antiviral artificial cell of the embodiment, since the virus is disinfected by the heat after it is entrapped by the artificial cytoskeleton, it is possible to reliably perform neutralization of the virus. Also, since the virus neutralization is externally controllable, it is possible to exhibit the antiviral action at a desired part of a human body or the like and at the most appropriate timing. Further, it is possible to prevent generation of a drug resistance virus and to prepare a countermeasure for an emerging virus in a short time.

Though the embodiments of this invention have been described in the foregoing, this invention is not limited to the embodiments, and it is possible to modify the embodiments in the scope of technical ideas of this invention. For example, though the nanoparticle 13 is rotated by way of the external oscillation in the foregoing description, the method of rotating the nanoparticle 13 is not limited thereto, and it is also preferable to use a magnetic material having a magnetic spin as the nanoparticle 13. In this case, the nanoparticle 13 is not decentered. When the nanoparticle 13 has the magnetic spin, it is possible to perform the treatment of affected area more efficiently since it is possible to guide the antiviral artificial cell 10 with the use of an external DC magnetic filed and to rotate the nanoparticle 13 with the use of an external rotating magnetic field. Also, the physical excitation means for rotating the nanoparticle is not limited to the oscillation and the rotating magnetic field, and it is possible to use an electromagnetic wave and light as the physical excitation means.

What is claimed is:

1. An antiviral artificial cell, comprising:
an artificial cytoskeleton;
an artificial cytomembrane wrapping the artificial cytoskeleton; and
a nanoparticle rotatably retained on the artificial cytomembrane and having a surface for capturing a virus.

2. The antiviral artificial cell according to claim 1,
wherein the artificial cytoskeleton comprises an electromagnetic wave absorber that generates heat to neutralize the virus when externally irradiated with an electromagnetic wave.

3. The antiviral artificial cell according to claim 1,
wherein the surface includes a concavo-convex part for fitting the virus thereon.

4. The antiviral artificial cell according to claim 1,
wherein the nanoparticle is decentered.

5. The antiviral artificial cell according to claim 1,
wherein the nanoparticle includes a magnetic spin.

6. The antiviral artificial cell according to claim 1,
wherein the nanoparticle comprises at least one of a metal, a semiconductor, a resin, and a ceramic.

7. The antiviral artificial cell according to claim 2,
wherein the electromagnetic wave absorber comprises a carbon nanotube.

8. A method for neutralizing a virus, comprising:
arranging a nanoparticle rotatably on an artificial cytomembrane that wraps an artificial cytoskeleton;
capturing a virus outside the artificial cytomembrane on a surface of the nanoparticle;
entrapping the captured virus into the artificial cytoskeleton; and
heating the artificial cytoskeleton by an irradiation of electromagnetic wave.
